(19) United States
(12) Patent Application Publication
(10) Pub. No.: US 2014/0276074 A1
(43) Pub. Date: Sep. 18, 2014

(54) Flexible Driveshafts with Bi-Directionally Balanced Torsional Stiffness Properties

(71) Applicant: W.L. Gore & Associates, Inc., (US)

(72) Inventor: Clifford P. Warner, West Chester, PA (US)

(73) Assignee: W.L. Gore & Associates, Inc., Newark, DE (US)

(21) Appl. No.: 13/800,515

(22) Filed: Mar. 13, 2013

(51) Int. Cl. F16C 1/02; A61B 8/00

(52) U.S. Cl. CPC F16C 1/02; A61B 8/445
USPC 600/459; 464/183

(57) ABSTRACT

This document provides flexible driveshaft devices. For example, this document provides flexible driveshaft devices that have bi-directionally balanced torsional stiffness properties. In some embodiments, the flexible driveshaft devices provided herein are utilized in medical device systems, such as endoluminal medical device systems. For example, in some embodiments the flexible driveshaft devices provided herein are utilized in endoluminal ultrasonic catheter systems.

### TECHNICAL FIELD

This document relates to flexible driveshafts. For example, this document relates to flexible driveshafts with bi-directionally balanced torsional stiffness properties that are suited for use in medical device systems.

### BACKGROUND

A flexible driveshaft is a long slender shaft used to transmit torque that is also flexible in bending. Said another way, flexible driveshafts can transmit torque along a tortuous route. Consequently, flexible driveshafts are well-suited for use in some medical devices, such as various kinds of catheter-based medical devices and systems.

Catheters can perform a variety of diagnostic or interventional medical procedures such as imaging, ablation, cutting, abrading, and biopsy, to provide a few examples. Some catheter-based medical devices are intended for use within a vessel, cavity, or duct of a patient. Some catheter-based medical devices utilize imaging technologies such as intravascular ultrasound, intracardiac echocardiography, optical coherence tomography, endoscopy, and angioscopy.

Flexible driveshafts are used widely in many types of medical devices. Some additional examples include medullary canal reamers, dental drills, orthopedic devices, arthroscopic and endoscopic instruments, and bronchoscopy devices. Such medical devices can be used for neuro-surgical, gastrointestinal, urinary, intrauterine, ear-nose-throat, orthopedic procedures, and the like.

Some currently available flexible driveshafts possess very different torsional stiffness properties when twisted in clockwise versus counterclockwise directions. This imbalance presents a problem for applications that require precise control of the angular position at the end of a long flexible driveshaft, and particularly when the application requires rotations in both directions.

### SUMMARY

This document provides flexible driveshaft devices. For example, this document provides flexible driveshaft devices that have bi-directionally balanced torsional stiffness properties. In some embodiments, the flexible driveshaft devices provided herein can be effectively utilized in medical device systems, such as catheter-based medical device systems. In some embodiments, the catheter-based medical device systems are inserted in a bodily vessel, cavity, or duct and manipulated by a portion that extends outside the body. For example, in some embodiments the flexible driveshaft devices provided herein can be effectively utilized in endoluminal catheter-based medical device systems, including but not limited to intravascular catheter-based medical device systems. Such catheter-based medical device systems can include acoustic imaging catheters, delivery systems for implantable devices such as stent grafts, and any other medical device and medical device system that utilizes flexible driveshaft devices.

In general, one aspect of this document features a flexible driveshaft for use in catheters. The driveshaft comprises a first helically wound layer of filars wound in a first direction, and a second helically wound layer of filars wound in a second direction. The second helically wound layer of filars are wrapped around an outer diameter of the first helically wound layer of filars. The second direction is generally opposite to the first direction. A ratio of the driveshaft's torsional stiffness in a clockwise direction of rotation to the driveshaft's torsional stiffness in a counterclockwise direction of rotation is within a range of about 1:2 to about 2:1. The driveshaft has an overall outer diameter of less than about 10 millimeters.

In various implementations, the driveshaft may further comprise a solid core. The first helically wound layer of filars may be wrapped around an outer diameter of the solid core. The driveshaft may further comprise a tubular core. The first helically wound layer may be wrapped around an outer diameter of the tubular core. The tubular core may comprise a metal or a polymeric material. The tubular core may define multiple lumens within the tubular core. An inner diameter of the first helically wound layer of filars may define a hollow central area of the driveshaft. The driveshaft may further comprise a third helically wound layer of filars wound in the first direction. The third helically wound layer of filars may be wrapped around an outer diameter of the second helically wound layer of filars. The driveshaft may further comprise a fourth helically wound layer of filars wound in the second direction. The fourth helically wound layer of filars may be wrapped around an outer diameter of the third helically wound layer of filars. The filars may be metallic wires. The ratio of the driveshaft's torsional stiffness in a clockwise direction of rotation to the driveshaft's torsional stiffness in a counterclockwise direction of rotation may be within a range of about 1:1.5 to about 1.5:1. The ratio of the driveshaft's torsional stiffness in a clockwise direction of rotation to the driveshaft's torsional stiffness in a counterclockwise direction of rotation may be about 1:1. The overall outer diameter of the driveshaft may be less than about 4 millimeters. The overall outer diameter of the driveshaft may be less than about 2 millimeters. The driveshaft has an overall length, and the driveshaft may have an overall length to overall outer diameter ratio that is greater than about 100:1. The driveshaft may have an overall length to overall outer diameter ratio that is greater than about 500:1.

In general, another aspect of this document features a catheter-based ultrasound system. The catheter-based ultrasound system comprises a flexible driveshaft and an ultrasound array coupled to the driveshaft. The flexible driveshaft comprises a first helically wound layer of filars wound in a first direction, and a second helically wound layer of filars wound in a second direction and wrapped around an outer diameter of the first helically wound layer of filars. The second direction is generally opposite to the first direction. A ratio of the driveshaft's torsional stiffness in a clockwise direction of rotation to the driveshaft's torsional stiffness in a counterclockwise direction of rotation is within a range of about 1:2 to about 2:1. The driveshaft has an overall outer diameter of less than about 10 millimeters.

In various implementations of the catheter-based ultrasound system, the driveshaft may further comprise a solid core, wherein the first helically wound layer may be wrapped around an outer diameter of the solid core. The driveshaft may further comprise a tubular core, wherein the first helically wound layer may be wrapped around an outer diameter of the tubular core. The tubular core may comprise a metal or polymer material. The tubular core may define multiple lumens within the tubular core. An inner diameter of the first helically wound layer of filars may define a hollow central area of the driveshaft. The driveshaft may further comprise a third helically wound layer of filars wound in the first direction and wrapped around an outer diameter of the second helically wound layer. The driveshaft may further comprise a fourth helically wound layer of filars wound in the second direction and wrapped around an outer diameter of the third helically wound layer of filars. The ratio of the driveshaft's torsional stiffness in a clockwise direction of rotation to the driveshaft's torsional stiffness in a counterclockwise direction of rotation may be within a range of about 1.5:1 to about 1:1.5. The ratio of the driveshaft's torsional stiffness in a clockwise direction of rotation to the driveshaft's torsional stiffness in a counterclockwise direction of rotation may be about 1:1. The overall outer diameter of the driveshaft may be less than about 4 millimeters. The overall outer diameter of the driveshaft may be less than about 2 millimeters. The filars may be metal wires. The driveshaft has an overall length, and the driveshaft may have an overall length to overall outer diameter ratio that is greater than about 100:1. The driveshaft may have an overall length to overall outer diameter ratio that is greater than about 500:1.

Particular embodiments of the subject matter described in this specification can be implemented so as to realize one or more of the following advantages. In some embodiments, medical device systems using a flexible driveshaft with bi-directionally balanced torsional stiffness properties provide a low profile catheter-based system with accurate and repeatable two-way tip panning. In some embodiments, medical device systems using a flexible driveshaft with bi-directionally balanced torsional stiffness properties provide improved system performance and higher accuracy of results. In some embodiments, the steering angle and tip rotation of the distal end of a catheter-based system can be repeatably and quantifiably located, thereby facilitating the use of a recipe approach for performing a medical procedure. In some embodiments, the enhanced accuracy and ease-of-use of systems using a flexible driveshaft with bi-directionally balanced torsional stiffness properties may eliminate the need for the operation of the system by a specialist, reduce the complexity or difficulty of use of the system by the user, reduce procedure times, reduce patient discomfort, and reduce costs.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIGS. 1A-1C illustrate perspective views of an example embodiment of a catheter-based ultrasonic imaging device.

FIG. 2 illustrates a perspective cut-away view of an example flexible driveshaft with bi-directionally unbalanced torsional stiffness properties.

FIG. 3 illustrates a cross-sectional view of the example flexible driveshaft of FIG. 2.

FIG. 4 is a graph showing plots of the angle of rotation of an actuator versus the angle of rotation of a tip for (i) a flexible driveshaft with bi-directionally unbalanced torsional stiffness and (ii) a flexible driveshaft with bi-directionally balanced torsional stiffness.

FIG. 5 illustrates a perspective cut-away view of an example flexible driveshaft with bi-directionally balanced torsional stiffness.

FIG. 6 illustrates a cross-sectional view of the example flexible driveshaft of FIG. 5.

FIG. 7 illustrates a perspective cut-away view of another example flexible driveshaft with bi-directionally balanced torsional stiffness.

FIG. 8 illustrates a perspective cut-away view of another example flexible driveshaft with bi-directionally balanced torsional stiffness.

FIG. 9 illustrates a perspective cut-away view of another example flexible driveshaft with bi-directionally balanced torsional stiffness.

FIG. 10 is a graph of torsion test data.

Like reference numbers and designations in the various drawings indicate like elements.

### DETAILED DESCRIPTION

This document provides flexible driveshaft devices that have bi-directionally balanced torsional stiffness properties. In some embodiments, the flexible driveshafts provided herein are applicable to catheter-based medical devices. Some such catheter-based medical devices are tubular devices that may be inserted in a bodily vessel, cavity, or duct and manipulated by a portion that extends outside the body. In some embodiments, the flexible driveshafts provided herein are applicable, without limitation, to medical devices used for neuro-surgical, gastrointestinal, urinary, intrauterine, intracardiac, ear-nose-throat, orthopedic, endoscopic, endoluminal, and intravascular procedures. In some embodiments, the flexible driveshaft devices provided herein can be utilized in devices that perform diagnostic or interventional procedures such as imaging, ablation, thrombectomy, atherectomy, cutting, abrading, biopsy, and delivery systems for implantable medical devices such as stents, stent grafts, and occluders to provide a few examples. The use of the flexible driveshaft devices provided herein is described in detail below in the context of an intravascular ultrasonic imaging catheter system as a non-limiting example implementation.

With reference to FIGS. 1A-1C, an example ultrasound catheter 100 includes an actuator control handle 110, a flexible driveshaft 120, and a distal tip 130 with an ultrasound array 131. The driveshaft 120 exits the distal end of the control handle 110 and extends distally to the distal tip 130 that is coupled to the distal end of the driveshaft 120. In some embodiments, the driveshaft 120 is within the lumen of a sheath, and the driveshaft 120 is free to turn within the lumen while the sheath is restrained from such turning. In some embodiments, no sheath with a lumen to house the driveshaft is used, and therefore the outer surface of the driveshaft 120 is exposed. In some embodiments, the outer surface of the driveshaft is a metallic or polymeric outer covering that is integral with the driveshaft. In some embodiments, no such covering is used. An image plane 132 is emitted from the ultrasound array 131 at the distal tip 130. The image plane 132 is schematically represented.

The usage of catheter-based medical devices, such as the example ultrasound catheter 100, is well known to those of skill in the art. In general, to use a catheter-based medical device the distal end of the catheter is inserted in a bodily vessel, cavity, or duct of a patient. The catheter is then maneuvered through the anatomy of the patient as needed to position a working portion of the catheter, such as the distal tip 130 with the ultrasound array 131, at a target location. Guidewires are used in some cases. Additional guidance catheters are used in some cases. Radiographic visualization and/or other types of visualization techniques are used in some cases. Typically the catheter can be manipulated by a clinician at a proximal portion of the catheter that extends outside the patient's body. With the working portion of the catheter properly positioned within the bodily vessel, cavity, or duct of a patient, the procedure may then be performed.

It should be noted that the example ultrasound catheter 100 is not necessarily drawn to scale. For example, the length of the driveshaft 120 can be longer than depicted. In some embodiments, the length of the driveshaft 120 is about 20 cm to about 70 cm, about 60 cm to about 120 cm, about 100 cm to about 150 cm, about 130 cm to about 180 cm, about 160 cm to about 220 cm, or about 200 cm to about 260 cm or longer. Driveshafts having any practical length are envisioned within the scope of this document.

The ultrasound catheter 100 is provided as an example to illustrate an implementation of the flexible driveshaft devices having bi-directionally balanced torsional stiffness properties as provided herein. However, as mentioned above, many other implementations of the flexible driveshaft devices are also envisioned. Such implementations include other medical devices, as well as other devices and systems that are not directly medically related.

The actuator control handle 110 includes a panning knob 112 and a steering knob 116. In some embodiments, the panning knob 112 and steering knob 116 are operated by a clinician to control the position and orientation of the distal portion of the driveshaft 120 and the distal tip 130. In some embodiments, the movements of the panning knob 112 and steering knob 116 are powered manually. In some embodiments, the movements are powered using, for example, motors that are controlled by a clinician, an automation system, or a combination thereof. In some embodiments, the motors are coupled at a proximal end of the drive shaft 120 located outside the patient's body. In some embodiments, a micro-motor is coupled to the driveshaft 120 at a location that can be positioned internal to the patient's body during a medical procedure.

In some embodiments, endoluminal imaging systems use a controlled sweeping or rotary motion of the image plane 132 at the distal tip 130 to create 3D or 4D images. For example, in some embodiments a stepper motor drive system controlled by an automation system is used to controllably drive the rotation (e.g., the direction and speed) of the driveshaft 120. In some such embodiments, by rotating the driveshaft 120 using the stepping motor, the image plane 132 can potentially be driven in a particular known, controllable, and repeatable sweep pattern. In some embodiments, various other types of motor driven systems are used.

In some embodiments, the image plane 132 is repeatedly swept back and forth through a prescribed angle of rotation and at a prescribed speed. For example, in some embodiments the image plane 132 is swept, or wobbled, back and forth at a specified rate of speed through a 90 degree arc of rotation. In such embodiments, the driveshaft 120 repeatedly reverses its direction of rotation. In some embodiments a 360° scan of a body lumen is performed. In some embodiments a 720° scan of a body lumen is performed. A variety of other prescribed patterns of sweeping and oscillating the distal tip 130 via the driveshaft 120 can be performed. As described further below, the flexible driveshaft devices provided herein facilitate improved accuracy, predictability, and repeatability of catheter-based medical devices, including endoluminal imaging systems that sweep and oscillate a distal tip 130 through a prescribed pattern of rotation at a prescribed speed.

The panning knob 112 is coupled, directly or indirectly, to the flexible driveshaft 120. The panning knob 112 is used to effect a rotary or panning movement by the distal tip 130. As the panning knob 112 is rotated, torque is transmitted via the driveshaft 120 from the panning knob 112 to the distal tip 130. As a result, the rotation of the panning knob 112 tends to cause a rotation of the distal tip 130 and, consequently, the ultrasound array 131. For example, the panning knob 112 can be rotated by the clinician in clockwise and/or counterclockwise directions as represented by arrow 114. Such rotation will tend to cause a rotation of the distal tip 130 as represented by arrow 134. FIGS. 1A and 1B represent, in comparison to each other, the rotation of the panning knob 112 and the resulting panning movement of the image plane 132 of the ultrasound array 131 at the distal tip 130.

While a rotation of the panning knob 112 (manually and/or using a motor-driven system) tends to cause a panning movement of the distal tip 130, in some instances the ratio of the rotation of the panning knob 112 to the rotation of the distal tip 130 is not 1:1. In other words, in some cases a rotation of the panning knob 112 by a certain number of degrees will result in a lesser or greater number of degrees of rotation of the distal tip 130. That is the case because the flexible shaft 120 is likely to have some torsional elasticity. In general, and as described further below, as the torsional stiffness of the flexible shaft 120 is increased, the ratio between the rotation of the panning knob 112 and the rotation of the distal tip 130 trends toward a 1:1 ratio.

In some embodiments of catheter-based medical devices that use a flexible driveshaft, it is desirable to have a ratio between the rotation of the driveshaft at the actuator and the rotation at the tip that is about 0.5:1, about 0.6:1, about 0.7:1, about 0.8:1, about 0.9:1, about 1:0.5, about 1:0.6, about 1:0.7, about 1:0.8, about 1:0.9, or about 1:1. That is the case because such a ratio will allow a clinician or automation system to exert predictable and repeatable control over the rotatory movement and resulting position of the tip portion. For example, in the context of the example ultrasound catheter 100, it is desirable for a rotation of the panning knob 112 by a certain amount to induce a predictable and repeatable amount of rotation of the ultrasound array 131 at the distal tip 130. In some embodiments, the ideal ratio is 1:1, or substantially close to 1:1. In that case, the quality of the ultrasound images are enhanced, the ultrasound imaging procedure provides more accurate results, and the device is easier for clinicians to use—with accompanying shortened procedure times, lower costs, and less patient discomfort.

It is also desirable in some embodiments for the ratio between the rotation at the panning knob 112 in comparison to the rotation at the distal tip 130 to be approximately equal in both directions of rotation, i.e., clockwise and counterclockwise. That way, the clinician will be able to exert predictable and repeatable control over the rotatory movement and position of the distal tip 130 in both directions of panning rotation.

As depicted in FIG. 1C, the steering knob 116 is used to manipulate the distal portion of the driveshaft 120. In some embodiments, a linear movement of the steering knob 116 in the proximal or distal direction as represented by arrow 118 induces the distal portion of the driveshaft 120 to bend transversely as represented by arrow 124. This motion is facilitated by a driveshaft 120 that is flexible in bending. In this manner, a clinician can actively steer the ultrasound catheter 100 through the anatomy of a patient which may be tortuous in nature. In some embodiments, radiopaque markers 122 are included on the ultrasound catheter 100 to enable radiographic visualization while steering the ultrasound catheter 100 through the anatomy of the patient.

In some embodiments, it is beneficial for catheter-based medical devices to be laterally flexible along all or part of the catheter. For example, in some embodiments, steering of catheter-based medical devices is performed by transverse bending of the catheter. In some embodiments in which steering is not performed, laterally flexibility of the catheter is beneficial (e.g., during catheter insertion) so that the catheter can conform to the tortuous non-linear anatomy of the patient. Consequently, such catheters, or portions of such catheters, are benefitted by having driveshafts with lateral flexibility, i.e., being flexible in bending. That is, a driveshaft that is laterally stiff will be resistant to bending and steering, while a driveshaft that is laterally flexible will be more amenable to bending and steering.

In general, for at least the reasons described above, it is desirable in some embodiments for the driveshaft of catheter-based medical devices, such as the example ultrasound catheter 100, to be a suitable combination of: (1) torsionally stiff, (2) equally torsionally stiff in both directions of rotation, and (3) laterally flexible.

A flexible driveshaft construction that may be used in attempt to meet the above properties consists of layers of helically wound wires (or “filars”) surrounding a core. The helically wound filars usually include at least two layers of filars, with the layers being wound in opposite directions. One set of filars is layered on top of another set of filars. The benefit of this construct is that it creates a driveshaft with a lower bending stiffness compared to a solid shaft of equivalent diameter, while providing a relatively high torsional stiffness. However, one shortcoming of this driveshaft construction is that it commonly has very different torsional stiffness properties in the clockwise versus the counterclockwise twist directions.

With reference to FIGS. 2 and 3, an example flexible driveshaft 200 with bi-directionally unbalanced torsional stiffness properties is depicted. FIG. 2 provides a cut-away perspective view, and FIG. 3 provides an orthogonal cross-sectional view at section 3-3. The flexible driveshaft 200 includes a solid core 210, a first helically wound layer of filars 220, and a second helically wound layer of filars 230. The first helically wound layer of filars 220 is wrapped around and overlays the core 210, and the second helically wound layer of filars 230 is wrapped around overlays the first helically wound layer of filars 220. The first helically wound layer of filars 220 and the second helically wound layer of filars 230 are wound in opposite directions. The first helically wound layer of filars 220 can be described as being left-wound (i.e., counterclockwise). The second helically wound layer of filars 230 can be described as being right-wound (i.e., clockwise). However, in some embodiments, the first helically wound layer of filars 220 are right-wound and the second helically wound layer of filars 230 are left-wound.

In the context of FIG. 2, when a clockwise torque, as represented by arrow 202, is applied to the driveshaft 200 the driveshaft 200 primarily transmits the torque via the core 210 and the second helically wound layer of filars 230. The second helically wound layer of filars 230 transmits the clockwise torque 202 because its layer of right-wound filars tend to get tightened by the clockwise torque 202. By contrast, the clockwise torque 202 tends to loosen the left-wound first helically wound layer of filars 220. Therefore, relatively less clockwise torque 202 is transmitted by the first helically wound layer of filars 220. On that basis, it can be said that in the context of FIG. 2 the right-wound filars are active when subjected to a clockwise torque, and that the left-wound filars are effectively inactive.

When a counterclockwise torque, as represented by arrow 204, is applied to driveshaft 200, the driveshaft 200 primarily transmits the torque via the core 210 and the first helically wound layer of filars 220. The first helically wound layer of filars 220 transmits the counterclockwise torque 204 because its layer of left-wound filars tend to get tightened by the counterclockwise torque 204. By contrast, the counterclockwise torque 204 tends to loosen the right-wound second helically wound layer of filars 230. Therefore, relatively less counterclockwise torque 204 is transmitted by the second helically wound layer of filars 230. On that basis, it can be said that in the context of FIG. 2 the left-wound filars are active when subjected to a counterclockwise torque, and that the right-wound filars are effectively inactive.

The torsional stiffness of a driveshaft can be expressed as:

S=GJ,

where:
S is the torsional stiffness;
G is the shear modulus of the driveshaft material; and
J is the polar moment of inertia of the driveshaft.

Based on the above, the formula for torsional stiffness of a flexible driveshaft having a core and layers of filars is then:

S=S_{core}+Σ(GJ)_{active filar layers},

where:
S_{core} is the torsional stiffness of the core; and
(GJ)_{active filar layers} is the shear modulus of the material of the active filar layer(s) multiplied by the polar moment of inertia of the active filar layer(s).

A driveshaft with bi-directionally balanced torsional stiffness properties has substantially equal torsional stiffness values in the clockwise and counterclockwise directions. This is stated as:

S_{cw}=S_{ccw}; or

S_{core}+Σ(GJ)_{right-wound filar layers}=S_{core}+Σ(GJ)_{left-wound filar layers}; or

Σ(GJ)_{right-wound filar layers}=Σ(GJ)_{left-wound filar layers}.

As shown in FIG. 3, orthogonal cross-sections of helically wound cylindrical filars are approximately elliptical. The polar moment of inertia of an ellipse through its centroid is expressed by:

Jₑₗₗᵢₚₛₑ=(π/4)(a³b+b³a)

where:
a is the radius of the major diameter of the ellipse; and
b is the radius of the minor diameter of the ellipse.

The parallel axis theorem states that the polar moment of inertia about an axis parallel to an axis through the centroid, about which a moment was calculated is expressed by:

J_{parallel axis}=J_{centroid}+Ad²

where:
A is the cross sectional area; and
d is the distance between the axes.

The area, A, of an ellipse equals nab, so the polar moment of inertia for a single filar can be described by:

J_{filar}=(π/4)(a³b+b³a)+πabR²

where:
R is the radius from the center of the driveshaft to the center of the filars for a given layer of filars.

The radius of the minor axis of the cross-section of a filar, b, is simply the radius of the filar. The radius of the major axis can be determined from the radius of the centerline of the layer of filars and the number of filars, N, used in that layer by:

a=(2πR)/N

Substituting (2πR)/N for a in the equation above for J_{filar}, and multiplying by the number of filars in a layer, one obtains an expression for the polar moment of inertia for a layer having N filars, with filar diameters of 2b, at a centerline radius R:

J_{layer}=N{(π/4)[(2πR/N)³b+b³(2πR/N)]+(2π²R³b)/N}

As illustrated further below, this equation for the polar moment of inertia for a layer of filars can be used to calculate whether a flexible driveshaft design has bi-directionally balanced or bi-directionally unbalanced torsional stiffness properties.

Still referring to FIGS. 2 and 3, the example flexible driveshaft 200 has the following characteristics. The core 210 is 0.660 mm in diameter and is made of nitinol. The first helically wound layer of filars 220 has twelve (12) filars that are 0.178 mm in diameter and are made of stainless steel. The second helically wound layer of filars 230 has eleven (11) filars that are 0.254 mm in diameter and are also made of the same type of stainless steel.

When applying the equation above for the polar moment of inertia for a layer of filars, J_{layer}, to the flexible driveshaft 200, the ratio of the clockwise polar moment of inertia to the counterclockwise polar moment of inertia can be calculated to be about 5:1.

Since in this example all the filars are made of the same type of stainless steel, the shear modulus G is consistent. Therefore, the ratio of the clockwise torsional stiffness to the counterclockwise torsional stiffness is also about 5:1. In other words, the example flexible driveshaft 200 is five (5) times stiffer when rotated in the clockwise direction than it is when rotated in the counterclockwise direction. In the context of a flexible driveshaft used in a catheter-based medical device, such a discrepancy can hinder a clinician from being able to exert predictable and repeatable control over the rotatory movement and position of the distal tip of the catheter in both directions of rotation.

With reference to FIG. 4, a graph 400 of tip angle 410 versus actuator angle 420 exemplifies the differences between flexible driveshafts with bi-directionally unbalanced torsional stiffness properties (plot 430) in comparison to flexible driveshafts with bi-directionally balanced torsional stiffness properties (plot 440). Graph 400 relates to, for example, catheter-based medical devices, such as the example ultrasound catheter 100, that use a flexible driveshaft. In general, graph 400 shows that control of the distal tip from the actuator is much less accurate using a driveshaft with bi-directionally unbalanced torsional stiffness properties (plot 430) in comparison to the control of the bi-directionally balanced driveshafts (plot 440) as provided herein.

Plot 430 illustrates that driveshafts with bi-directionally unbalanced torsional stiffness properties exhibit a number of performance deficiencies. First, portions 432 and 434 reflect instances where substantial rotations of the panning knob at the actuator result in virtually no rotation of the tip. Portion 432, for example, indicates that the panning knob has been rotated from about 360 degrees to about 150 degrees, but that virtually no rotation of the tip has occurred. Similarly, portion 434 indicates that the panning knob has been rotated from about −360 degrees to about −50 degrees, but that virtually no rotation of the tip has occurred. Those instances provide examples of when the clinician does not have accurate control over the tip angle. As a result, the medical procedure is potentially more time-consuming, expensive, and the potential for patient discomfort is increased.

In addition, plot 430 includes portion 436 where, while the actuator is minimally rotated, the tip rotates from about −270 degrees to about −130 degrees. Further still, portions 436 and 438 are asymmetrical. These instances provide further examples of when the clinician does not have accurate control over the tip angle.

Unlike the driveshafts with bi-directionally unbalanced torsional stiffness properties, the bi-directionally balanced driveshafts as provided herein provide substantially better controllability of the tip angle, as illustrated by plot 440. For example, plot 440 does not include instances where the panning knob of the actuator is rotated and no rotation of the tip takes place. Nor does plot 440 include instances where the panning knob of the actuator is minimally rotated but a large change in the tip angle results. Further, plot 440 is generally symmetrical. Therefore, the bi-directionally balanced driveshafts as provided herein provide substantially better controllability of the tip angle.

With reference to FIGS. 5 and 6, an example flexible driveshaft 500 with bi-directionally balanced torsional stiffness properties is depicted. FIG. 5 provides a cut-away perspective view, and FIG. 6 provides an orthogonal cross-sectional view at section 6-6. The flexible driveshaft 500 is a three-layer design. The flexible driveshaft 500 includes a hollow core 510, a first helically wound layer of filars 520, a second helically wound layer of filars 530, and a third helically wound layer of filars 540. The first helically wound layer of filars 520 is wrapped around and overlays the core 510; the second helically wound layer of filars 530 is wrapped around and overlays the first helically wound layer of filars 520; and the third helically wound layer of filars 540 is wrapped around and overlays the second helically wound layers of filars 530. The adjacent layers of helically wound filars are wound in opposite directions. That is, the first helically wound layer of filars 520 is left-wound, the second helically wound layer of filars 530 is right-wound, and the third helically wound layer of filars 540 is left-wound.

In some embodiments, the directions of the winds are reversed in comparison to the example flexible driveshaft 500. For example, in some embodiments the first helically wound layer of filars 520 is right-wound, the second helically wound layer of filars 530 is left-wound, and the third helically wound layer of filars 540 is right-wound. In some embodiments, two or more adjacent layers of filars are wound in the same direction, rather than being wound in opposite directions. For example, in some such embodiments two or more adjacent layers of filars are both right-wound, or two or more adjacent layers of filars are both left-wound. All combinations and subcombinations of filar layer wind directions are envisioned within the scope of this disclosure.

The core 510 can have a variety of construction configurations. In some embodiments, the core 510 is a metallic material, e.g., nitinol, stainless steel, titanium, titanium alloys (e.g., titanium beta 3), chrome cobalt alloys, precipitation hardened stainless steels, ultra-high-strength steels (e.g., ferrium S53), or another suitable metal or metal alloy. In some embodiments, the core 510 is a polymeric material. For example, in some such embodiments the core is a thermoplastic polymer that is expanded above its glass transition temperature by pressure in order to bring the core 510 into direct contact with the first helically wound layer of filars 520. In general, the material selection for the core can be based on the parameters desired for the flexible driveshaft. A metallic core may be a desirable core material in some embodiments of flexible driveshafts because of its generally high yield strength in bending, so as to resist plastic deformation in bending at greater than a minimum bend radius. A polymeric core may be a desirable core material in some embodiments of flexible driveshafts because of its ductility and relative low elastic modulus.

In some embodiments, the core 510 has different cross-sectional geometries (size or shape) at different portions along the axial length of the core 510. In some embodiments, the core 510 has a cross-sectional geometry other than a circle, e.g., an ovular, square, triangular, or another suitable shape. In some embodiments, the cross-sectional geometry of the core 510 can be suited to transmitting torque via the flexible driveshaft 500. For example, in some embodiments an ovular cross-section, or another noncircular cross-sectional shape, can be used to enhance the transmission of torque using the flexible driveshaft 500.

In some embodiments, the core 510 is solid, rather than hollow as shown. In some embodiments of flexible driveshafts, no core is used in the finished flexible driveshaft device (e.g., refer to FIG. 8). In some such embodiments, the inside of the first helically wound layer of filars defines a lumen or hollow central area of the driveshaft.

The individual filars used to construct the layers of helically wound filars 520, 530, and 540 can have a variety of configurations. In some embodiments, the filars are a metallic material, e.g., nitinol, stainless steels (e.g., 316LVM), titanium, titanium alloys (e.g., titanium beta 3), or another suitable metal or metal alloy. In some embodiments, the filars are or include graphite, Kevlar, or a polymeric material. In some embodiments, the filars can be woven, rather than wound, layers. In some embodiments, individual filars can comprise multiple strands of material that are twisted, woven, or otherwise coupled together to form a filar. In some embodiments, the filars have different cross-sectional geometries (size or shape) at different portions along the axial length of the driveshaft 500. In some embodiments, the filars have a cross-sectional geometry other than a circle, e.g., an ovular, square, triangular, or another suitable shape. In some embodiments, the filars of one layer have different configurations (e.g., their size, shape, construction, and material) than the filars of another layer. While in some embodiments the filars of a given layer each have the same configuration, in some embodiments, one or more filars of a given layer have different configurations than one or more of the other filars of the given layer. In some embodiments, a combination or subcombination of such factors are used in a single flexible driveshaft embodiment.

Referring to FIG. 6, the core 510 and layers of helically wound filars 520, 530, and 540 overlay on each other to define an overall outer diameter 542 of the flexible driveshaft 500. In some embodiments, the overall outer diameter 542 is within a range of about 0.5 mm to about 2 mm, or about 1 mm to about 4 mm, or about 3 mm to about 8 mm, or about 7 mm to about 12 mm, or more than about 12 mm.

When a clockwise torque, as represented by arrow 502, is applied to driveshaft 500, the driveshaft 200 primarily transmits the torque via the core 210 and the second helically wound layer of filars 530. The second helically wound layer of filars 530 transmits the clockwise torque 502 because its right-wound filars tend to get tightened by the clockwise torque 502. By contrast, the clockwise torque 502 tends to loosen the first helically wound layer of filars 520 and the third helically wound layer of filars 540. Therefore, relatively less clockwise torque 502 is transmitted by the left-wound first helically wound layer of filars 520 or the left-wound third helically wound layer of filars 540. On that basis, it can be said that, in the context of FIG. 5, right-wound filars are active when subjected to a clockwise torque, and that the left-wound filars are effectively inactive.

When a counterclockwise torque, as represented by arrow 504, is applied to driveshaft 500, the driveshaft 500 primarily transmits the torque via the core 510, the first helically wound layer of filars 520, and the third helically wound layer of filars 540. The first helically wound layer of filars 520 and the third helically wound layer of filars 540 transmit the counterclockwise torque 504 because their left-wound filars tend to get tightened by the counterclockwise torque 504. By contrast, the counterclockwise torque 504 tends to loosen the second helically wound layer of filars 530. Therefore, relatively less counterclockwise torque 504 is transmitted by the right-wound second helically wound layer of filars 530. On that basis, it can be said that, in the context of FIG. 5, left-wound filars are active when subjected to a counterclockwise torque, and that the right-wound filars are effectively inactive.

Applying the formulas from above, it is known that for example flexible driveshaft 500 to have bi-directionally balanced torsional stiffness properties, the following relationship applies:

Σ(GJ)_{right-wound filar layers}=Σ(GJ)_{left-wound filar layers}; or

(GJ)_{second layer}=(GJ)_{first layer}+(GJ)_{third layer}

The example flexible driveshaft 500 can be designed to have substantially bi-directionally balanced torsional stiffness properties using several different filar configuration combinations. One example embodiment is configured as follows (this example is based on all filars being constructed of the same material, e.g., stainless steel (“SS”)):
the first filar layer 520 has 12 filars that are 0.127 mm in diameter;
the second filar layer 530 has 9 filars that are 0.254 mm in diameter; and
the third filar layer 520 has 18 filars that are 0.101 mm in diameter.

Alternatively, one could produce a three-layer flexible driveshaft with substantially bi-directionally balanced torsional stiffness properties using dissimilar types of filar materials (thereby affecting the shear modulus, G). One potential benefit of this would be to use a larger filar diameter for the outermost layer, which could deter the outermost filars from springing back, or unwrapping. One example embodiment using dissimilar types of filar materials is configured is as follows:
the first filar layer 520 has 12 SS filars at 0.127 mm in diameter;
the second filar layer 530 has 9 SS filars at 0.229 mm in diameter; and
the third filar layer 540 has 18 Titanium Beta 3 alloy filars at 0.152 mm in diameter.

While the example flexible driveshaft 500 is a three-layer design, a two-layer driveshaft design with substantially bi-directionally balanced torsional stiffness properties can also be constructed. One example embodiment is configured as follows:
the first filar layer has 7 SS filars at 0.318 mm in diameter; and
the second filar layer has 18 SS filars at 0.114 mm in diameter.

The filar material can also be varied within a two-layer driveshaft. One example embodiment of a two-layer driveshaft with substantially bi-directionally balanced torsional stiffness properties, with dissimilar filar material is configured as follows:
the first filar layer has 7 SS filars at 0.259 mm in diameter; and
the second filar layer has 18 Titanium Beta 3 alloy filars at 0.173 mm in diameter.

Several different flexible driveshafts with substantially bi-directionally balanced torsional stiffness properties can be configured, and the configurations described above are provided as non-limiting illustrative examples. Design parameters including but not limited to the filar diameters used for each filar layer, the number of filars in each filar layer, the filar wrap angle, the cross-sectional shape of the filars, the shear modulus of the material the filars are made from, and the number of filar layers can be selected as desired. When designing a flexible driveshaft with substantially bi-directionally balanced torsional stiffness properties any of those types of design parameters may be manipulated in order to design a flexible driveshaft with the desired properties for a particular implementation. In general, the design parameters with the greatest relative effect on torsional stiffness are the filar diameters and the shear modulus of the filar material.

FIGS. 7-9 illustrate additional example flexible driveshafts 700, 800, and 900 that can be configured to have bi-directionally balanced torsional stiffness properties. Example flexible driveshaft 700 is a three-layer design like that of FIG. 5, but with a solid core 710. Example flexible driveshaft 800 is a three-layer design like that of FIG. 5, but without a core. Example flexible driveshaft 900 is a four-layer design.

As example flexible driveshaft 900 illustrates, flexible driveshafts with substantially bi-directionally balanced torsional stiffness properties can be configured with four, or more, layers of filars. The flexible driveshaft 900 includes a solid core 910, a first left-wound helically wound layer of filars 920, a second right-wound helically wound layer of filars 930, a third left-wound helically wound layer of filars 940, and a fourth right-wound helically wound layer of filars 950. The first helically wound layer of filars 920 is wrapped around and overlays the core 910; the second helically wound layer of filars 930 is wrapped around and overlays the first helically wound layer of filars 920; the third helically wound layer of filars 940 is wrapped around and overlays the second helically wound layers of filars 930; and the fourth helically would layer of filars 950 is wrapped around and overlays the third helically wound layer of filars 940. In other embodiments, the filars can be wound in several other directional configurations and combinations of configurations.

Applying the formulas from above, for driveshaft 900 to have bi-directionally balanced torsional stiffness properties the following relationship applies:

Σ(GJ)_{right-wound filar layers}=Σ(GJ)_{left-wound filar layers}; or

(GJ)_{second layer}+(GJ)_{fourth layer}=(GJ)_{first layer}+(GJ)_{third layer}

The example flexible driveshaft 900 can be designed to have substantially bi-directionally balanced torsional stiffness properties using several different filar configuration combinations. One example embodiment is configured as follows (this example is based on all filars being constructed of the same material, e.g., stainless steel (“SS”)):
the first filar layer 920 has 14 filars that are 0.076 mm in diameter;
the second filar layer 930 has 18 filars that are 0.076 mm in diameter;
the third filar layer 940 has 10 filars that are 0.254 mm in diameter; and
the fourth filar layer 950 has 18 filars that are 0.102 mm in diameter.

Alternatively, one could produce a four-layer flexible driveshaft 900 with substantially bi-directionally balanced torsional stiffness properties where the filar material is varied. One such example embodiment is configured is as follows:
the first filar layer 920 has 14 SS filars at 0.127 mm in diameter;
the second filar layer 930 has 18 SS filars at 0.229 mm in diameter;
the third filar layer 940 has 10 SS filars at 0.203 mm in diameter; and
the fourth filar layer 950 has 18 Titanium Beta 3 alloy filars at 0.152 mm in diameter.

The response of a shaft placed in torsion can be expressed as:

T=(□/L)S,

where:
T is the applied Torque;
□ is the angle of twist of the shaft;
L is the length of the shaft; and
S is the stiffness previously described.

FIG. 10 shows a graph 1000 of torsion test data, where the applied torque is plotted versus □/L. The slopes of the plotted curves represent the torsional stiffness of the shafts. The data shown was generated from two different shaft constructions.

The first shaft construction tested, representing a commercially available design, (corresponding to plot 1010) was an unbalanced two-layer construction configured as follows:
the core was 0.660 mm in diameter and made of nitinol;
the first filar layer had twelve filars that were 0.178 mm in diameter and were made of stainless steel; and
the second filar layer had eleven filars that were 0.254 mm in diameter and were also made of the same type of stainless steel.

The second shaft construction tested (corresponding to plot 1020) was a balanced three-layer construction built to the following specifications:
the core was 0.508 mm in diameter and made of nitinol;
the first filar layer had twelve filars that were 0.127 mm in diameter and were made of stainless steel;
the second filar layer had nine filars that were 0.254 mm in diameter and were also made of the same type of stainless steel; and
the third filar layer had eighteen filars that were 0.102 mm in diameter and were also made of the same type of stainless steel.

The plot for the two-layer shaft construction 1010 has a significant inflection at about 0.2 degrees/mm. The ratio of the slopes on either side of the inflection is about 4.7:1. This matches very well to the predictions one would make according to the calculations provided above.

In contrast, the plot for the three-layer shaft construction 1020 with the balanced design is approximately a straight line. The ratio of slopes on either side of zero is about 1.15:1, which matches very well with the predicted ratio of 1.10:1 as would be predicted according to the calculations provided above.

While this specification contains many specific implementation details, these should not be construed as limitations on the scope of any devices, methods, and systems discussed herein, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Particular embodiments of the subject matter have been described. Other embodiments are within the scope of the following claims.

What is claimed is:

1. A flexible driveshaft for use in catheters, wherein the driveshaft comprises:
a first helically wound layer of filars wound in a first direction; and
a second helically wound layer of filars wound in a second direction and wrapped around an outer diameter of the first helically wound layer of filars, the second direction being generally opposite to the first direction,
wherein a ratio of the driveshaft's torsional stiffness in a clockwise direction of rotation to the driveshaft's torsional stiffness in a counterclockwise direction of rotation is within a range of about 1:2 to about 2:1, and wherein the driveshaft has an overall outer diameter of less than about 10 millimeters.

2. The driveshaft of claim 1, further comprising a solid core, wherein the first helically wound layer of filars is wrapped around an outer diameter of the solid core.

3. The driveshaft of claim 1, further comprising a tubular core, wherein the first helically wound layer is wrapped around an outer diameter of the tubular core.

4. The driveshaft of claim 3, wherein the tubular core comprises a metal or a polymeric material.

5. The driveshaft of claim 3, wherein the tubular core defines multiple lumens within the tubular core.

6. The driveshaft of claim 1, wherein an inner diameter of the first helically wound layer of filars defines a hollow central area of the driveshaft.

7. The driveshaft of claim 1, further comprising a third helically wound layer of filars wound in the first direction and wrapped around an outer diameter of the second helically wound layer of filars.

8. The driveshaft of claim 7, further comprising a fourth helically wound layer of filars wound in the second direction and wrapped around an outer diameter of the third helically wound layer of filars.

9. The driveshaft of claim 1, wherein the filars are metallic wires.

10. The driveshaft of claim 1, wherein the ratio is within a range of about 1:1.5 to about 1.5:1.

11. The driveshaft of claim 1, wherein the ratio is about 1:1.

12. The driveshaft of claim 1, wherein the overall outer diameter of the driveshaft is less than about 4 millimeters.

13. The driveshaft of claim 1, wherein the overall outer diameter of the driveshaft is less than about 2 millimeters.

14. The driveshaft of claim 1, wherein the driveshaft has an overall length, and wherein the driveshaft has an overall length to overall outer diameter ratio that is greater than about 100:1.

15. The driveshaft of claim 1, wherein the driveshaft has an overall length, and wherein the driveshaft has an overall length to overall outer diameter ratio that is greater than about 500:1.

16. A catheter-based ultrasound system, comprising:
a flexible driveshaft comprising:
a first helically wound layer of filars wound in a first direction; and
a second helically wound layer of filars wound in a second direction and wrapped around an outer diameter of the first helically wound layer of filars, the second direction being generally opposite to the first direction,
wherein a ratio of the driveshaft's torsional stiffness in a clockwise direction of rotation to the driveshaft's torsional stiffness in a counterclockwise direction of rotation is within a range of about 1:2 to about 2:1, and wherein the driveshaft has an overall outer diameter of less than about 10 millimeters; and
an ultrasound array coupled to the driveshaft.

17. The catheter-based ultrasound system of claim 16, wherein the driveshaft further comprises a solid core, and wherein the first helically wound layer is wrapped around an outer diameter of the solid core.

18. The catheter-based ultrasound system of claim 16, wherein the driveshaft further comprises a tubular core, and wherein the first helically wound layer is wrapped around an outer diameter of the tubular core.

19. The catheter-based ultrasound system of claim 18, wherein the tubular core comprises a metal or polymer material.

20. The catheter-based ultrasound system of claim 18, wherein the tubular core defines multiple lumens within the tubular core.

21. The catheter-based ultrasound system of claim 16, wherein an inner diameter of the first helically wound layer of filars defines a hollow central area of the driveshaft.

22. The catheter-based ultrasound system of claim 16, wherein the driveshaft further comprises a third helically wound layer of filars wound in the first direction and wrapped around an outer diameter of the second helically wound layer.

23. The catheter-based ultrasound system of claim 22, wherein the driveshaft further comprises a fourth helically wound layer of filars wound in the second direction and wrapped around an outer diameter of the third helically wound layer of filars.

24. The catheter-based ultrasound system of claim 16, wherein the ratio is within a range of about 1.5:1 to about 1:1.5.

25. The catheter-based ultrasound system of claim 16, wherein the ratio is about 1:1.

26. The catheter-based ultrasound system of claim 16, wherein the overall outer diameter of the driveshaft is less than about 4 millimeters.

27. The catheter-based ultrasound system of claim 16, wherein the overall outer diameter of the driveshaft is less than about 2 millimeters.

28. The catheter-based ultrasound system of claim 16, wherein the filars are metal wires.

29. The catheter-based ultrasound system of claim 16, wherein the driveshaft has an overall length, and wherein the driveshaft has an overall length to overall outer diameter ratio that is greater than about 100:1.

30. The catheter-based ultrasound system of claim 16, wherein the driveshaft has an overall length, and wherein the driveshaft has an overall length to overall outer diameter ratio that is greater than about 500:1.
